SEQUENCE LISTING (6 sequences)

SEQ ID NO: 1 - DNA, 17 bp, Artificial Sequence
  PCR forward primer
agcctcgcctttgccga

SEQ ID NO: 2 - DNA, 15 bp, Artificial Sequence
  PCR reverse primer
ctggtgcctggggcg

SEQ ID NO: 3 - DNA, 22 bp, Artificial Sequence
  PCR probe
ccgccgcccgtccacacccgcc

SEQ ID NO: 4 - DNA, 20 bp, Artificial Sequence
  PCR forward primer
cgytggatgcgnttycatga

SEQ ID NO: 5 - DNA, 22 bp, Artificial Sequence
  PCR reverse primer
cttagacgccatcatcattyac

SEQ ID NO: 6 - DNA, 20 bp, Artificial Sequence
  PCR probe
agatcgcggtctcctgtcca

(19) United States
(12) Patent Application Publication
(10) Pub. No.: US 2016/0121332 A1
(43) Pub. Date: May 5, 2016

(54) NUCLEIC ACID AMPLIFICATION REACTION APPARATUS AND NUCLEIC ACID DETECTION METHOD

(71) Applicant: Seiko Epson Corporation, Tokyo (JP)

(72) Inventor: Akemi YAMAGUCHI, Matsumoto (JP)

(21) Appl. No.: 14/926,678

(22) Filed: Oct. 29, 2015

(30) Foreign Application Priority Data
Oct. 31, 2014 (JP) 2014-222570

(51) Int. Cl. B01L 7/00; C12Q 1/68

(52) U.S. Cl. CPC B01L 7/525; C12Q 1/6846

(57) ABSTRACT

A nucleic acid amplification reaction apparatus includes: a fitting section capable of fitting a nucleic acid amplification reaction vessel comprising a nucleic acid amplification reaction mixture; temperature adjustment sections which adjust the temperatures of first and second regions of the nucleic acid amplification reaction vessel, respectively; and a driving mechanism which switches the location of the first and second regions; the nucleic acid amplification reaction mixture includes first and second primer pairs, the reaction apparatus performs a thermal cycle by setting the temperatures of the first and second regions to a first temperature and a second temperature, respectively, and thereafter performs a thermal cycle by setting the temperatures of the first and second regions to a third temperature and a fourth temperature, respectively.

### BACKGROUND

1. Technical Field

The present invention relates to a nucleic acid amplification reaction apparatus and a nucleic acid detection method.

2. Related Art

In recent years, as a result of development of technologies utilizing genes, medical treatments utilizing genes such as gene diagnosis or gene therapy have been drawing attention. In addition, many methods using genes in determination of breed varieties or breed improvement have also been developed in agricultural and livestock industries. As technologies for utilizing genes, nucleic acid amplification technologies such as a PCR (Polymerase Chain Reaction) method have been widely used.

In nucleic acid amplification technologies, as a method of simultaneously detecting a plurality of genes, multiplex PCR has been known (see, for example, JP-A-2010-207220).

In multiplex PCR, however, in the case where a plurality of genes in one reaction mixture are detected, a plurality of primer pairs for the respective genes are used, and therefore, in order to distinguish the genes to be amplified, different types of fluorescent dyes whose number is the same as the number of the genes and detection systems for the respective fluorescent dyes are needed, and also, reactions are sometimes not sufficient due to interference among respective primers, etc. Accordingly, it was difficult to establish the reaction conditions.

### SUMMARY

An advantage of some aspects of the invention is to provide a nucleic acid amplification reaction apparatus and a nucleic acid detection method capable of detecting each of a plurality of genes in one reaction system accurately and simply in a short time.

The invention can be implemented as the following forms.

A nucleic acid amplification reaction apparatus according to an aspect of the invention includes: a fitting section capable of fitting a nucleic acid amplification reaction vessel filled with a nucleic acid amplification reaction mixture and a liquid which has a specific gravity smaller than that of the nucleic acid amplification reaction mixture and is immiscible with the nucleic acid amplification reaction mixture; a first temperature adjustment section which adjusts the temperature of a first region of the nucleic acid amplification reaction vessel; a second temperature adjustment section which adjusts the temperature of a second region of the nucleic acid amplification reaction vessel; and a driving mechanism which switches over between a first arrangement in which the first region is located lower than the second region in the direction of the gravitational force and a second arrangement in which the second region is located lower than the first region in the direction of the gravitational force, wherein for the nucleic acid amplification reaction mixture, which contains a first primer pair for amplifying a first nucleic acid and a second primer pair for amplifying a second nucleic acid, and in which the annealable temperature range of the first primer pair to the first nucleic acid and the annealable temperature range of the second primer pair to the second nucleic acid overlap by 10° C. or less, a first thermal cycle is performed by adjusting the temperature of the first region to a first temperature and adjusting the temperature of the second region to a second temperature which is lower than the first temperature, and thereafter, a second thermal cycle is performed by adjusting the temperature of the first region to a third temperature and adjusting the temperature of the second region to a fourth temperature which is lower than the third temperature and different from the second temperature. The second temperature may be higher than the fourth temperature. A temperature difference between the second temperature and the optimal annealing temperature of the first primer pair to the first nucleic acid may be 5° C. or less, or 1° C. or less. A temperature difference between the fourth temperature and the optimal annealing temperature of the second primer pair to the second nucleic acid may be 5° C. or less, or 1° C. or less. The first thermal cycle may be a thermal cycle in which a two-stage temperature change between the first temperature and the second temperature is repeated, and at the second temperature, the first nucleic acid may be amplified and the second nucleic acid may not be amplified, or the second thermal cycle may be a thermal cycle in which a two-stage temperature change between the third temperature and the fourth temperature is repeated, and at the fourth temperature, the second nucleic acid may be amplified and the first nucleic acid may not be amplified.

A nucleic acid detection method according to another aspect of the invention uses a nucleic acid amplification reaction apparatus including: a fitting section capable of fitting a nucleic acid amplification reaction vessel filled with a nucleic acid amplification reaction mixture and a liquid which has a specific gravity smaller than that of the nucleic acid amplification reaction mixture and is immiscible with the nucleic acid amplification reaction mixture; a first temperature adjustment section which adjusts the temperature of a first region of the nucleic acid amplification reaction vessel; a second temperature adjustment section which adjusts the temperature of a second region of the nucleic acid amplification reaction vessel; and a driving mechanism which switches over between a first arrangement in which the first region is located lower than the second region in the direction of the gravitational force and a second arrangement in which the second region is located lower than the first region in the direction of the gravitational force, wherein for the nucleic acid amplification reaction mixture, which contains a first primer pair for amplifying a first nucleic acid and a second primer pair for amplifying a second nucleic acid, and in which the annealable temperature range of the first primer pair to the first nucleic acid and the annealable temperature range of the second primer pair to the second nucleic acid overlap by a predetermined temperature or less, the method includes: performing a first thermal cycle by setting the temperature of the first region to a first temperature and setting the temperature of the second region to a second temperature which is lower than the first temperature; and performing, after the first thermal cycle, a second thermal cycle by setting the temperature of the first region to a third temperature and setting the temperature of the second region to a fourth temperature which is lower than the third temperature and different from the second temperature. The annealable temperature ranges may overlap by 5° C. or less or may not overlap with each other. The optimal annealing temperature of the first primer pair to the first nucleic acid and the optimal annealing temperature of the second primer pair to the second nucleic acid may be different by 3° C. or more. The nucleic acid amplification reaction apparatus may further include a fluorescence detection section, and the fluorescence detection section may monitor fluorescence emitted from the nucleic acid amplification reaction mixture while the nucleic acid amplification reaction apparatus is performing the first thermal cycle and the second thermal cycle.

According to the aspects of the invention, a nucleic acid amplification reaction apparatus capable of determining the presence or absence of each of a plurality of genes accurately and simply in a short time can be provided. Specifically, according to the aspects of the invention, it is only necessary to prepare one fluorescence detection system and one reaction mixture, and therefore, the operation is simple and the cost can be reduced. Further, a dispensing operation is not needed, and therefore, an error due to the dispensing operation does not occur. Further, the inhibition of amplification reactions due to interference among respective primers caused by simultaneous amplification of a plurality of nucleic acids, which was a problem when a plurality of nucleic acids are amplified in one reaction mixture, does not occur. In addition, in the case of monitoring fluorescence brightness, the presence or absence of the amplification of a nucleic acid can be determined by a change in the fluorescence brightness, and therefore, an additional melting curve analysis or an electrophoresis operation is not needed, and thus, the determination can be achieved in a short time.

### BRIEF DESCRIPTION OF THE DRAWINGS

The invention will be described with reference to the accompanying drawings, wherein like numbers reference like elements.

FIG. 1 is a cross-sectional view of a nucleic acid amplification reaction vessel according to one embodiment of the invention. The arrow g indicates the direction of the gravitational force.

FIGS. 2A and 2B are perspective views of an elevating type PCR apparatus according to one embodiment of the invention. FIG. 1A shows a state in which a lid is closed and FIG. 1B shows a state in which the lid is opened.

FIG. 3 is an exploded perspective view of a main body of the elevating type PCR apparatus according to one embodiment of the invention.

FIGS. 4A and 4B are cross-sectional views schematically showing the cross section taken along the line A-A of FIG. 2A of the main body of the elevating type PCR apparatus according to one embodiment of the invention. FIG. 4A shows a first arrangement and FIG. 4B shows a second arrangement.

FIG. 5 shows curves representing the annealable temperature ranges of respective primer pairs according to Comparative Example.

FIG. 6 shows curves representing the annealable temperature ranges of respective primer pairs in the invention according to one embodiment of the invention.

FIG. 7 shows fluorescence brightness curves representing the amplification of respective genes according to one embodiment of the invention and Comparative Example.

### DESCRIPTION OF EXEMPLARY EMBODIMENTS

The object, characteristics, and advantages of the invention as well as the idea thereof will be apparent to those skilled in the art from the description given herein, and the invention can be easily reproduced by those skilled in the art based on the description given herein. It is to be understood that the embodiments, specific examples, etc. of the invention described below are to be taken as preferred embodiments of the invention, and are presented for illustrative or explanatory purposes and are not intended to limit the invention. It is further apparent to those skilled in the art that various changes and modifications may be made based on the description given herein within the intent and scope of the invention disclosed herein.

### (1) Nucleic Acid Amplification Reaction Vessel

A nucleic acid amplification reaction vessel to be used in the method according to the invention is a nucleic acid amplification reaction vessel which has a hermetically sealed vessel containing a reaction mixture and a liquid having a specific gravity different from that of the reaction mixture and phase-separated from the reaction mixture, wherein the reaction mixture is in the form of a liquid droplet and the liquid contains an oil and an additive.

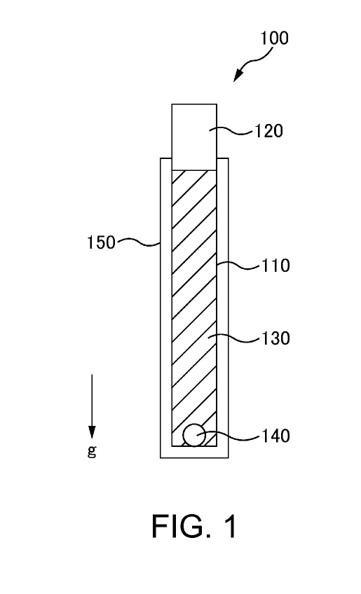

FIG. 1 is a cross-sectional view of a nucleic acid amplification reaction vessel 100. FIG. 1 shows a state in which a reaction mixture is placed in the nucleic acid amplification reaction vessel.

The nucleic acid amplification reaction vessel 100 to be used in the invention is configured to include a vessel 150 and a sealing section 120. The size and shape of the nucleic acid amplification reaction vessel 100 are not particularly limited, but may be designed in consideration of, for example, at least one of the amount of a liquid 130 which is immiscible with a reaction mixture 140, the thermal conductivity thereof, the shapes of the vessel 150 and the sealing section 120, and the ease of handling thereof.

The vessel 150 of the nucleic acid amplification reaction vessel 100 can be formed from a transparent material. According to this, the movement of the reaction mixture 140 in the vessel 150 can be observed from the outside of the nucleic acid amplification reaction vessel 100, or the vessel 150 can be used in an application in which the measurement or the like is performed from the outside of the vessel 150 such as real-time PCR. The term “transparent” as used herein refers to a condition in which the visibility can be ensured to such an extent that the reaction mixture 140 in the vessel 150 can be observed from the outside of the vessel 150, and it is not necessary that the entire nucleic acid amplification reaction vessel 100 should be transparent as long as this condition is met.

The application of the nucleic acid amplification reaction vessel 100 is not particularly limited, however, for example, in the case where the nucleic acid amplification reaction vessel 100 is used in an application with a fluorescence measurement such as real-time PCR, the vessel 150 is desirably formed from a material with a low autofluorescence. The vessel 150 is preferably formed from a material which can withstand heating in PCR. Further, the material of the vessel 150 is preferably a material, on which nucleic acids or proteins are less adsorbed, and which does not inhibit the enzymatic reaction by a polymerase or the like. The material which satisfies these conditions is not particularly limited, and for example, polypropylene, polyethylene, a cycloolefin polymer (for example, ZEONEX (registered trademark) 480R), a heat-resistant glass (for example, PYREX (registered trademark) glass), or the like, or a composite material thereof may be used, however, polypropylene is preferred.

In the nucleic acid amplification reaction vessel 100 shown in FIG. 1, the vessel 150 is formed into a cylindrical shape, and the direction of the center axis (the vertical direction in FIG. 1) coincides with the longitudinal direction. The vessel 150 used here is preferably a tube and may be a tube for a microcentrifuge or a tube designed for PCR. Since the vessel 150 has a shape with a longitudinal direction, in other words, an elongated shape, for example, in the case where the temperature of the nucleic acid amplification reaction vessel 100 is controlled so that regions having different temperatures are formed in the liquid 130 in the vessel 150 using an elevating type thermal cycler, which will be described later, the distance between the regions having different temperatures is easily increased, According to this, it becomes easy to control the temperature of the liquid 130 to be different from region to region in the vessel 150, and therefore, a thermal cycle suitable for PCR can be realized. The “elevating type thermal cycler” is an apparatus which performs a thermal cycle by forming at least two temperature regions in a liquid filled in the vessel 150 and allowing the reaction mixture 140 which is phase-separated from the liquid to move reciprocatingly between these temperature regions.

The shape of the vessel 150 is not particularly limited as long as it has a longitudinal direction, however, in the case where the vessel 150 is used for elevating type PCR, it is preferred that the shape is a substantially cylindrical shape and the ratio of the inner diameter D to the length L in the longitudinal direction is in the range of 1:5 to 5:20. It is more preferred that the inner diameter D is from 1.5 to 2 mm, and the length L is from 10 to 20 mm.

The vessel 150 has an opening section and the sealing section 120 which seals the opening section, and in the vessel 150, the reaction mixture 140 and the liquid 130 which has a specific gravity different from that of the reaction mixture 140 and is phase-separated from the reaction mixture 140 are contained. It is preferred that in the case where the opening section is sealed by the sealing section 120, air does not remain in the vessel 150. It is because if an air bubble remains in the vessel 150, the movement of the reaction mixture 140 may be hindered. The sealing section 120 can be formed from the same material as that of the vessel 150. The structure of the sealing section 120 may be any as long as it can hermetically seal the vessel 150, and can be a structure of, for example, a screw cap, a plug, an inlay, or the like. In FIG. 1, the sealing section 120 has a structure of a screw cap.

The nucleic acid amplification reaction mixture 140 (hereinafter also referred to as “reaction mixture 140”) may contain a reagent for a nucleic acid amplification reaction and a target nucleic acid to be amplified. Examples of the target nucleic acid include a DNA prepared from a specimen such as blood, urine, saliva, spinal fluid, or a tissue and a cDNA obtained by reverse transcription of an RNA prepared from any of the above specimens. The reagent for a nucleic acid amplification reaction may contain a primer pair for amplifying a target nucleic acid, a buffer, a polymerase, dNTPs, MgCl₂, a fluorescent label for detecting an amplification product of the target nucleic acid, and the like. In the case where RT-PCR is performed in the nucleic acid amplification reaction vessel 100, the reaction mixture 140 may contain a reverse transcriptase, a primer for reverse transcription, and the like. The DNA polymerase is not particularly limited, but is preferably a heat-resistant enzyme or an enzyme for use in PCR. There are a great number of commercially available products, for example, Taq polymerase, Tfi polymerase, Tth polymerase, modified forms thereof, and the like, however, a DNA polymerase capable of performing hot start is preferred. The concentration of dNTPs or a salt may be set to a concentration suitable for the enzyme to be used, however, the concentration of dNTPs may be set to generally 10 to 1000 μM, and preferably 100 to 500 μM, the concentration of Mg²⁺ may be set to generally 1 to 100 mM, and preferably 5 to 10 mM, and the concentration of Cl⁻ may be set to generally 1 to 2000 mM, and preferably 200 to 700 mM. The total ion concentration is not particularly limited, but may be higher than 50 mM, and is preferably higher than 100 mM, more preferably higher than 120 mM, further more preferably higher than 150 mM, and still further more preferably higher than 200 mM. The upper limit thereof is preferably 500 mM or less, more preferably 300 mM or less, and further more preferably 200 mM or less. Each oligonucleotide for the primer is used at 0.1 to 10 μM, and preferably at 0.1 to 1 μM. The fluorescent label for detecting the amplification product of the target nucleic acid is not particularly limited, and may be arbitrarily selected from commercially available products, for example, a fluorescent probe such as TaqMan (R) MGB probe (Applied Biosystems) an intercalater such as SYBR Green (R), etc. according to the purpose.

The reaction mixture 140 may further contain a surfactant. The surfactant is not particularly limited, however, examples thereof include NP-40, Triton X-100, and Tween 20. The concentration of the surfactant is not particularly limited, but is preferably a concentration which does not inhibit the nucleic acid amplification reaction, and may be from 0.001% to 0.1% or less, and is preferably from 0.002% to 0.02%, and most preferably from 0.005% to 0.01%. The surfactant may be a carry-over from a stock solution of the enzyme described above, however, a surfactant solution may be added to the reaction mixture 140 independently of the stock solution of the enzyme.

By using a liquid which is immiscible with the reaction mixture 140 as the liquid 130, when the reaction mixture 140 is placed in the vessel 150, the reaction mixture 140 and the liquid 130 are phase-separated from each other, and therefore, the reaction mixture 140 can be formed into a liquid droplet in the liquid 130. In this manner, the reaction mixture 140 is maintained in the form of a liquid droplet in the liquid 130.

The liquid 130 is preferably a liquid having a specific gravity smaller than that of the reaction mixture 140. In this case, when the reaction mixture 140 is placed in the liquid 130, the liquid droplet of the reaction mixture 140 has a specific gravity larger than that of the liquid 130, and therefore moves in the direction of the gravitational force by the action of the gravity. Further, the liquid 130 may be a liquid having a specific gravity larger than that of the reaction mixture 140. In this case, the liquid droplet of the reaction mixture 140 has a specific gravity smaller than that of the liquid 130, and therefore moves in the direction opposite to the direction of the gravitational force by the action of the gravity.

The liquid 130 preferably contains an oil, and for example, a silicone oil or a mineral oil can be used. Here, the “silicone” means an oiligomer or a polymer having a siloxane bond as a main skeleton. In this specification, among silicones, a silicone in the form of a liquid in a temperature range in which the silicone is used in a thermal cycling treatment is particularly referred to as “silicone oil”. Further, in this specification, an oil which is purified from petroleum and is in the form of a liquid in a temperature range in which the oil is used in a thermal cycling treatment is referred to as “mineral oil”. These oils have high stability against heat, and for example, products having a viscosity of 5×10³ Nsm⁻² or less are also easily available, and therefore, these oils are preferred for use in elevating type PCR.

Examples of the silicone oil include dimethyl silicone oils such as KF-96L-0.65cs, KF-96L-1cs, KF-96L-2cs, KF-96L-5cs (manufactured by Shin-Etsu Silicone Co., Ltd.), SH200 C FLUID 5 CS (manufactured by Dow Corning Toray Co, Ltd.), TSF451-5A, and TSF451-10 (manufactured by Momentive Performance Materials Japan LLC). Examples of the mineral oil include oils containing alkane having about 14 to 20 carbon atoms as a principal component, and specific examples thereof include n-tetradecane, n-pentadecane, n-hexadecane, n-heptadecane, n-octadecane, n-nonadecane, and n-tetracosane.

The liquid 130 may contain an additive. As the additive, a modified silicone oil such as X-22-160AS, X-22-3701E, KF-857, KF-859, KF-862, KF-867, KF-6017, or KF-8005 (Shin-Etsu Silicone Co., Ltd.), a silicone resin such as SR1000, SS4230, SS4267, or YR3370 (Momentive Performance Materials, Inc.), a fluoro-modified silicone resin such as XS66-C1191 (Momentive Performance Materials, Inc.), or the like, and other than these, a modified silicone oil such as TSF4703, TSF4708, XF42-05196, or XF42-05197 (Momentive Performance Materials, Inc.) can be used. The concentration of the additive is not particularly limited, but can be determined in consideration of the structure, material, shape, or the like of the vessel. For example, the concentration thereof is preferably 1% (v/v) or more and 50% (v/v) or less, more preferably 2% (v/v) or more and 20% (v/v) or less, and further more preferably 5% (v/v).

### (2) Configuration of Elevating Type Nucleic Acid Amplification Reaction Apparatus

In this embodiment, as the nucleic acid amplification reaction vessel to be used for performing a nucleic acid amplification reaction, a nucleic acid amplification reaction tube 100 in the form of a tube is used. Hereinafter, by taking PCR as one example of the nucleic acid amplification example, one example of an elevating type nucleic acid amplification reaction apparatus (hereinafter also referred to as “elevating type PCR apparatus”) suitable for the nucleic acid amplification reaction tube 100 will be described in detail.

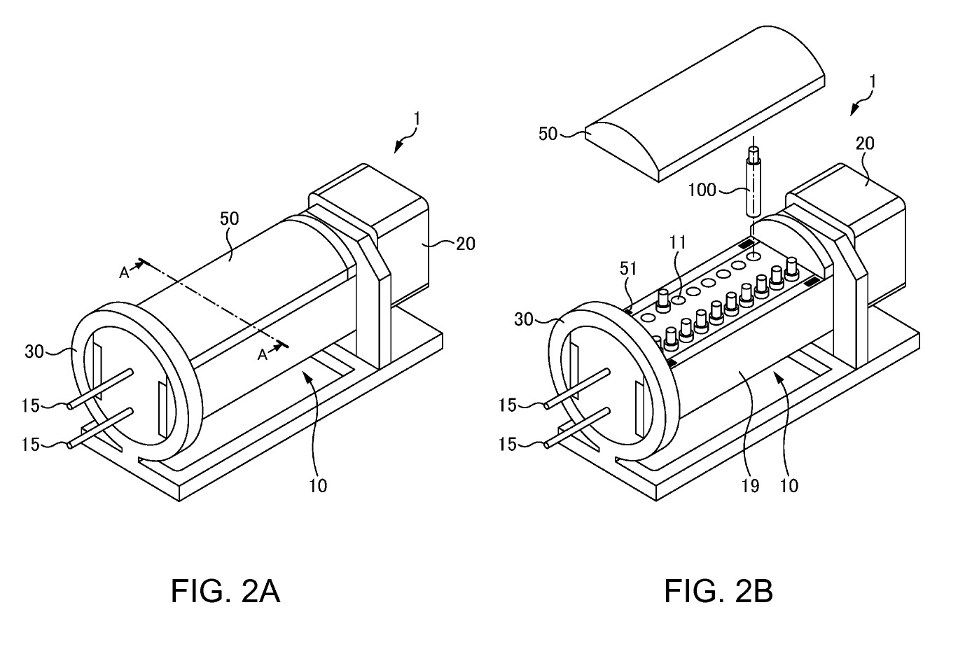

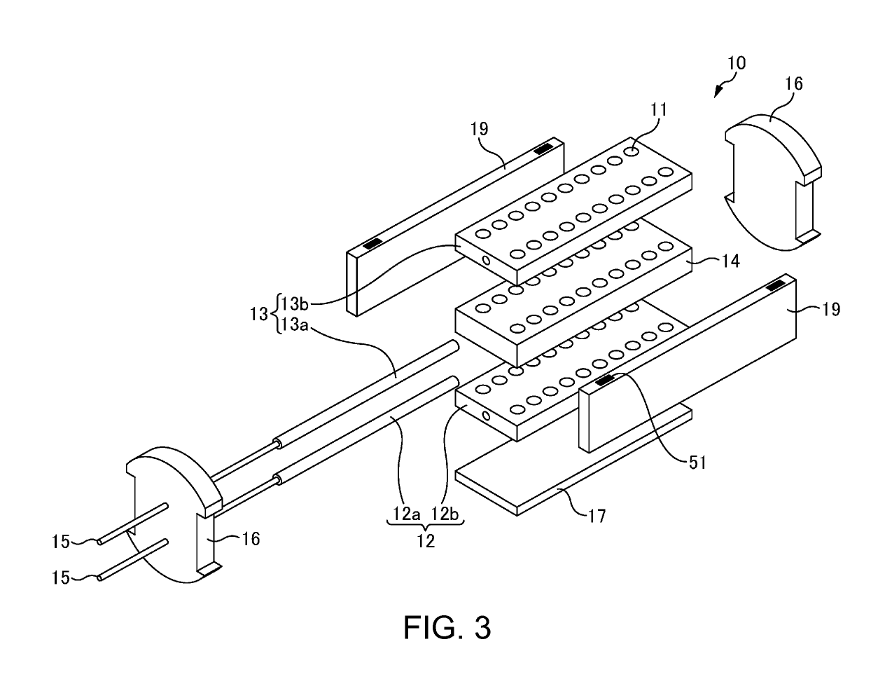

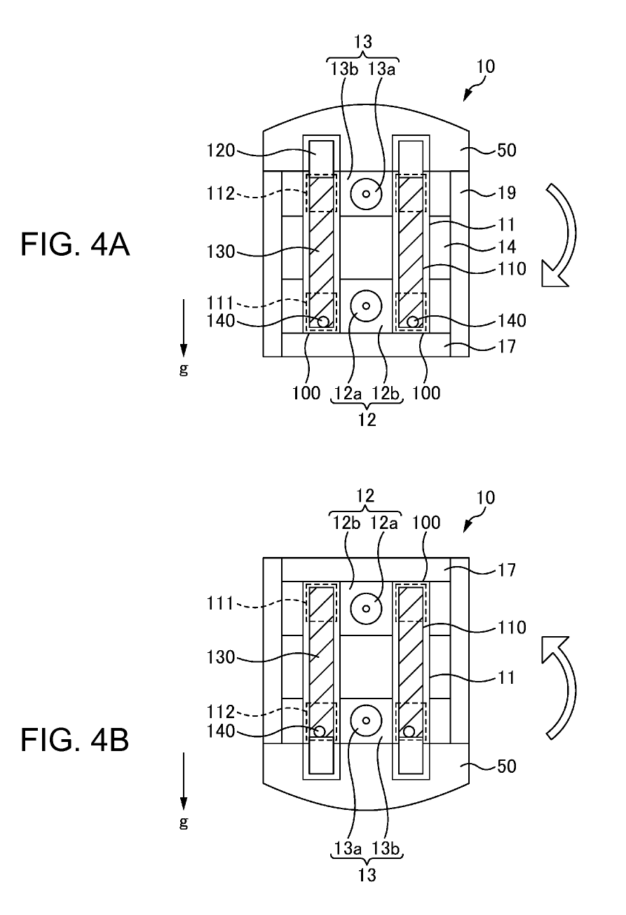

FIGS. 2A and 2B show one example of an elevating type PCR apparatus 1. FIG. 2A shows a state in which a lid 50 of the elevating type PCR apparatus 1 is closed, and FIG. 2B shows a state in which the lid 50 of the elevating type PCR apparatus 1 is opened and the nucleic acid amplification reaction tube 100 is fitted in a fitting section 11. FIG. 3 is an exploded perspective view of a main body 10 of the elevating type PCR apparatus 1 according to the embodiment. FIGS. 4A and 4B are cross-sectional views schematically showing the cross section taken along the line A-A of FIG. 2A of the main body 10 of the elevating type PCR apparatus 1 according to the embodiment.

This elevating type PCR apparatus 1 includes the main body 10 and a driving mechanism 20 as shown in FIG. 2A. As shown in FIG. 3, the main body 10 includes the fitting section 11, a first temperature adjustment section 12, and a second temperature adjustment section 13. A spacer 14 is provided between the first temperature adjustment section 12 and the second temperature adjustment section 13. In the main body 10 of this embodiment, the first temperature adjustment section 12 is disposed on the side of a bottom plate 17, and the second temperature adjustment section 13 is disposed on the side of the lid 50. In the main body 10 of this embodiment, the first temperature adjustment section 12, the second temperature adjustment section 13, and the spacer 14 are fixed by a flange 16, the bottom plate 17, and a fixing plate 19.

The fitting section 11 is configured such that the nucleic acid amplification reaction tube 100, which will be described later, is fitted therein. As shown in FIG. 2B and FIG. 3, the fitting section 11 of this embodiment has a slot structure in which the nucleic acid amplification reaction tube 100 is inserted and fitted, and is configured such that the nucleic acid amplification reaction tube 100 is inserted into a hole penetrating a first heat block 12b of the first temperature adjustment section 12, the spacer 14, and a second heat block 13b of the second temperature adjustment section 13. The number of the fitting sections 11 may be more than one, and in the example shown in FIG. 2B, twenty fitting sections 11 are provided for the main body 10.

This elevating type PCR apparatus 1 includes a structure in which the nucleic acid amplification reaction tube 100 is held at a predetermined position with respect to the first temperature adjustment section 12 and the second temperature adjustment section 13. More specifically, as shown in FIGS. 4A and 4B, in a flow channel 110 constituting the nucleic acid amplification reaction tube 100, which will be described later, the temperature of a first region 111 can be adjusted by the first temperature adjustment section 12 and the temperature of a second region 112 can be adjusted by the second temperature adjustment section 13. In this embodiment, a structure that defines the position of the nucleic acid amplification reaction tube 100 is the bottom plate 17, and as shown in FIG. 4A, by inserting the nucleic acid amplification reaction tube 100 to a position where the tube is in contact with the bottom plate 17, the nucleic acid amplification reaction tube 100 can be held at a predetermined position with respect to the first temperature adjustment section 12 and the second temperature adjustment section 13.

When the nucleic acid amplification reaction tube 100 is fitted in the fitting section 11, the first temperature adjustment section 12 adjusts the temperature of the first region 111 of the nucleic acid amplification reaction tube 100, which will be described later, to a first temperature. In the example shown in FIG. 4A, in the main body 10, the first temperature adjustment section 12 is disposed at a position where the first region 111 of the nucleic acid amplification reaction tube 100 is heated.

The first temperature adjustment section 12 may include a mechanism that generates heat and a member that transfers the generated heat to the nucleic acid amplification reaction tube 100. In the example shown in FIG. 3, the first temperature adjustment section 12 includes a first heater 12a and a first heat block 12b. In this embodiment, the first heater 12a is a cartridge heater and is connected to an external power source (not shown) through a conductive wire 15. The first heater 12a is inserted into the first heat block 12b, and the first heat block 12b is heated by heat generated by the first heater 12a. The first heat block 12b is a member that transfers heat generated by the first heater 12a to the nucleic acid amplification reaction tube 100. In this embodiment, the first heat block 12b is a block made of aluminum.

When the nucleic acid amplification reaction tube 100 is fitted in the fitting section 11, the second temperature adjustment section 13 adjusts the temperature of the second region 112 of the nucleic acid amplification reaction tube 100 to a second temperature different from the first temperature. In the example shown in FIG. 4A, in the main body 10, the second temperature adjustment section 13 is disposed at a position where the second region 112 of the nucleic acid amplification reaction tube 100 is heated. As shown in FIGS. 2A and 2B, the second temperature adjustment section 13 includes a second heater 13a and the second heat block 13b. The second temperature adjustment section 13 is configured in the same manner as the first temperature adjustment section 12 except that the region of the nucleic acid amplification reaction tube 100 to be heated and the heating temperature are different from those for the first temperature adjustment section 12.

In this embodiment, the temperatures of the first temperature adjustment section 12 and the second temperature adjustment section 13 are controlled by a temperature sensor (not shown) and a control section (not shown), which will be described later. The temperatures of the first temperature adjustment section 12 and the second temperature adjustment section 13 are preferably set so that the temperatures of the first and second regions of the nucleic acid amplification reaction tube 100 are adjusted to desired temperatures. In this embodiment, by controlling the first temperature adjustment section 12 at the first temperature and the second temperature adjustment section 13 at the second temperature, the first region 111 of the nucleic acid amplification reaction tube 100 can be heated to the first temperature, and the second region 112 can be heated to the second temperature. The temperature sensor in this embodiment is a thermocouple.

The driving mechanism 20 is a mechanism that controls the fitting section 11, the first temperature adjustment section 12, and the second temperature adjustment section 13, and by the driving mechanism, the arrangement of the first region and the second region is controlled. In this embodiment, the driving mechanism 20 includes a motor (not shown) and a drive shaft (not shown), and the drive shaft is connected to the flange 16 of the main body 10. The drive shaft in this embodiment is provided perpendicular to the longitudinal direction of the fitting section 11, and when the motor is activated, the main body 10 is rotated about the drive shaft as the axis of rotation.

The elevating type PCR apparatus 1 of this embodiment includes the control section (not shown). The control section controls at least one of the first temperature, the second temperature, a first period, a second period, and the cycle number of thermal cycles, which will be described later. In the case where the control section controls the first period or the second period, the control section controls the operation of the driving mechanism 20, thereby controlling the period in which the fitting section 11, the first temperature adjustment section 12, and the second temperature adjustment section 13 are held in a predetermined arrangement. The control section may have mechanisms different from item to item to be controlled, or may control all items collectively. However, the control section in the elevating type PCR apparatus 1 of this embodiment is an electronic control system and controls all of the above-mentioned items. The control section of this embodiment includes a processor such as a CPU (not shown) and a storage device such as an ROM (Read Only Memory) or an RAM (Random Access Memory). In the storage device, various programs, data, etc. for controlling the above-mentioned respective items are stored. Further, the storage device has a work area for temporarily storing data during treatment, treatment results, etc. of various treatments.

As shown in the example of FIG. 3 and FIG. 4A, in the main body 10 of this embodiment, the spacer 14 is provided between the first temperature adjustment section 12 and the second temperature adjustment section 13. The spacer 14 of this embodiment is a member that holds the first temperature adjustment section 12 or the second temperature adjustment section 13. In this embodiment, the spacer 14 is a heat insulating material, and in the example shown in FIG. 4A, the fitting section 11 penetrates the spacer 14.

The main body 10 of this embodiment includes the fixing plate 19. The fixing plate 19 is a member that holds the fitting section 11, the first temperature adjustment section 12, and the second temperature adjustment section 13. In the example shown in FIG. 2B and FIG. 3, two fixing plates 19 are fitted in the flanges 16, and the first temperature adjustment section 12, the second temperature adjustment section 13, and the bottom plate 17 are fixed by the fixing plates 19.

The elevating type PCR apparatus 1 of this embodiment includes the lid 50. In the example shown in FIG. 2A and FIG. 4A, the fitting section 11 is covered with the lid 50. The lid 50 may be fixed to the main body 10 by a fixing section 51. In this embodiment, the fixing section 51 is a magnet. As shown in the example of FIG. 2B and FIG. 3, a magnet is provided on a surface of the main body 10 which comes into contact with the lid 50. Although not shown in FIG. 2B and FIG. 3, a magnet is provided also for the lid 50 at a place where the magnet of the main body 10 comes into contact. When the fitting section 11 is covered with the lid 50, the lid 50 is fixed to the main body 10 by a magnetic force.

It is preferred that the fixing plate 19, the bottom plate 17, the lid 50, and the flange 16 are formed using a heat insulating material.

### (3) Thermal Cycling Treatment Using Elevating Type PCR Apparatus

FIGS. 4A and 4B are cross-sectional views schematically showing the cross section taken along the line A-A of FIG. 2A of the elevating type PCR apparatus 1. FIGS. 4A and 4B show a state in which the nucleic acid amplification reaction tube 100 is fitted in the elevating type PCR apparatus 1. FIG. 4A shows a first arrangement and FIG. 4B shows a second arrangement. Hereinafter, a thermal cycling treatment using the elevating type PCR apparatus 1 according to the embodiment in the case of using the nucleic acid amplification reaction tube 100 will be described.

As shown in the example of FIG. 1, the nucleic acid amplification reaction tube 100 according to the embodiment includes a flow channel 110 and a sealing section 120. The flow channel 110 is filled with a reaction mixture 140 and a liquid 130, which has a specific gravity smaller than that of the reaction mixture 140 and is immiscible with the reaction mixture 140, and sealed with the sealing section 120.

The flow channel 110 is formed such that the reaction mixture 140 moves in close proximity to opposed inner walls. Here, the phrase “opposed inner walls” of the flow channel 110 refers to two regions of a wall surface of the flow channel 110 having an opposed positional relationship. The phrase “in close proximity to” refers to a state in which the distance between the reaction mixture 140 and the wall surface of the flow channel 110 is close, and includes a case where the reaction mixture 140 is in contact with the wall surface of the flow channel 110. Therefore, the phrase “the reaction mixture 140 moves in close proximity to opposed inner walls” refers to that “the reaction mixture 140 moves in a state of being close in distance to both of the two regions of a wall surface of the flow channel 110 having an opposed positional relationship”, that is, the reaction mixture 140 moves along the opposed inner walls.

In the example shown in FIG. 1, the outer shape of the nucleic acid amplification reaction tube 100 is a cylindrical shape, and the flow channel 110 is formed in the direction of the center axis (the vertical direction in FIG. 1) therein. The shape of the flow channel 110 is a cylindrical shape having a circular cross section perpendicular to the longitudinal direction of the flow channel 110, that is, perpendicular to the direction in which the reaction mixture 140 moves in a region in the flow channel 110 (this cross section is defined as the “cross section” of the flow channel 110). Therefore, in the nucleic acid amplification reaction tube 100 of this embodiment, the opposed inner walls of the flow channel 110 are regions including two points on the wall surface of the flow channel 110 constituting the diameter of the cross section of the flow channel 110, and the reaction mixture 140 moves in the longitudinal direction of the flow channel 110 along the opposed inner walls.

The first region 111 of the nucleic acid amplification reaction tube 100 is a partial region of the flow channel 110 whose temperature is adjusted to the first temperature by the first temperature adjustment section 12. The second region 112 is a partial region of the flow channel 110, which is different from the first region 111, and whose temperature is adjusted to the second temperature by the second temperature adjustment section 13. In the nucleic acid amplification reaction tube 100 of this embodiment, the first region 111 is a region including one end portion in the longitudinal direction of the flow channel 110, and the second region 112 is a region including the other end portion in the longitudinal direction of the flow channel 110. In the example shown in FIGS. 4A and 4B, a region surrounded by the dotted line including an end portion on the proximal side of the sealing section 120 of the flow channel 110 is the second region 112, and a region surrounded by the dotted line including an end portion on the distal side of the sealing section 120 is the first region 111.

As shown in FIG. 1, the flow channel 110 contains the liquid 130 and a liquid droplet of the reaction mixture 140. The liquid 130 and the reaction mixture 140 are prepared according to the description of the (1) Nucleic Acid Amplification Reaction Vessel.

Hereinafter, with reference to FIGS. 4A and 4B, the thermal cycling treatment using the elevating type PCR apparatus 1 according to the embodiment will be described. In FIGS. 4A and 4B, the direction indicated by the arrow g (in the downward direction in the drawing) is the direction of the gravitational force. In this embodiment, a case where shuttle PCR (two-stage temperature PCR) is performed as an example of the thermal cycling treatment will be described. The respective steps described below show one example of the thermal cycling treatment, and according to need, the order of the steps may be changed, two or more steps may be performed sequentially or concurrently, or a step may be added.

The shuttle PCR is a method of amplifying a nucleic acid in a reaction mixture by subjecting the reaction mixture to a two-stage temperature treatment at a high temperature and a low temperature repeatedly. In the treatment at a high temperature, denaturation of a double-stranded DNA occurs and in the treatment at a low temperature, annealing (a reaction in which a primer is bound to a single-stranded DNA) and elongation (a reaction in which a complementary strand to the DNA is synthesized by using the primer as a starting point) occur.

In general, in shuttle PCR, the high temperature is a temperature between 80° C. and 100° C. and the low temperature is a temperature between 50° C. and 70° C. The treatments at the respective temperatures are performed for a predetermined period, and a period in which the reaction mixture is maintained at a high temperature is generally shorter than a period in which the reaction mixture is maintained at a low temperature. For example, the period of the treatment at a high temperature may be set to about 1 to 10 seconds, and the period of the treatment at a low temperature may be set to about 4 to 60 seconds, or a period longer than this range may be adopted depending on the condition of the reaction.

The appropriate period, temperature, cycle number (the number of repetitions of the treatment at a high temperature and the treatment at a low temperature) vary depending on the type or amount of a reagent to be used, and therefore, it is preferred to determine an appropriate protocol in consideration of the type of a reagent or the amount of the reaction mixture 140 before performing the reaction.

First, the nucleic acid amplification reaction tube 100 is fitted in the fitting section 11. In this embodiment, after the reaction mixture 140 is introduced into the flow channel 110 previously filled with the liquid 130, the nucleic acid amplification reaction tube 100 is sealed with the sealing section 120, and then fitted in the fitting section 11. The introduction of the reaction mixture 140 can be performed using a micropipette, an ink-jet dispenser, or the like. In a state in which the nucleic acid amplification reaction tube 100 is fitted in the fitting section 11, the first temperature adjustment section 12 is in contact with the nucleic acid amplification reaction tube 100 at a place including the first region 111 and the second temperature adjustment section 13 is in contact with the nucleic acid amplification reaction tube 100 at a place including the second region 112.

Here, the arrangement of the fitting section 11, the first temperature adjustment section 12, and the second temperature adjustment section 13 is the first arrangement. As shown in FIG. 4A, in the first arrangement, the first region 111 of the nucleic acid amplification reaction tube 100 is located in a lowermost portion of the flow channel 110 in the direction of the gravitational force. In the first arrangement, the first region 111 is located in a lowermost portion of the flow channel 110 in the direction of the gravitational force, and therefore, the reaction mixture 140 having a specific gravity larger than that of the liquid 130 is located in the first region 111. In this embodiment, after the nucleic acid amplification reaction tube 100 is fitted in the fitting section 11, the fitting section 11 is covered with the lid 50, and then the elevating type PCR apparatus 1 is operated.

Subsequently, the nucleic acid amplification reaction tube 100 is heated by the first temperature adjustment section 12 and the second temperature adjustment section 13. The first temperature adjustment section 12 and the second temperature adjustment section 13 adjust the temperatures of different regions of the nucleic acid amplification reaction tube 100 to different temperatures. That is, the first temperature adjustment section 12 adjusts the temperature of the first region 111 to the first temperature, and the second temperature adjustment section 13 adjusts the temperature of the second region 112 to the second temperature. According to this, a temperature gradient in which the temperature gradually changes between the first temperature and the second temperature is formed between the first region 111 and the second region 112 of the flow channel 110. Here, a temperature gradient in which the temperature decreases from the first region 111 to the second region 112 is formed. The thermal cycling treatment of this embodiment is shuttle PCR, and therefore, the first temperature is set to a temperature suitable for the denaturation of a double-stranded DNA, and the second temperature is set to a temperature suitable for annealing and elongation.

Since the arrangement of the fitting section 11, the first temperature adjustment section 12, and the second temperature adjustment section 13 is the first arrangement, when the nucleic acid amplification reaction tube 100 is heated, the reaction mixture 140 is heated to the first temperature. When the first period has elapsed, the main body 10 is driven by the driving mechanism 20, and the arrangement of the fitting section 11, the first temperature adjustment section 12, and the second temperature adjustment section 13 is switched over from the first arrangement to the second arrangement. The second arrangement is an arrangement in which the second region 112 is located in a lowermost portion of the flow channel 110 in the direction of the gravitational force. In other words, the second region 112 is a region located in a lowermost portion of the flow channel 110 in the direction of the gravitational force when the fitting section 11, the first temperature adjustment section 12, and the second temperature adjustment section 13 are placed in a predetermined arrangement different from the first arrangement. In the elevating type PCR apparatus 1 of this embodiment, under the control of the control section, the driving mechanism 20 rotatively drives the main body 10. When the flanges 16 are rotatively driven by the motor by using the drive shaft as the axis of rotation, the fitting section 11, the first temperature adjustment section 12, and the second temperature adjustment section 13 which are fixed to the flanges 16 are rotated. Since the drive shaft is a shaft extending in the direction perpendicular to the longitudinal direction of the fitting section 11, when the drive shaft is rotated by the activation of the motor, the fitting section 11, the first temperature adjustment section 12, and the second temperature adjustment section 13 are rotated. In the example shown in FIGS. 4A and 4B, the main body 10 is rotated at 180°. By doing this, the arrangement of the fitting section 11, the first temperature adjustment section 12, and the second temperature adjustment section 13 is switched over from the first arrangement to the second arrangement.

Here, the positional relationship between the first region 111 and the second region 112 in the direction of the gravitational force is opposite to that of the first arrangement, and therefore, the reaction mixture 140 moves from the first region 111 to the second region 112 by the action of the gravity. When the operation of the driving mechanism 20 is stopped after the arrangement of the fitting section 11, the first temperature adjustment section 12, and the second temperature adjustment section 13 has reached the second arrangement, the fitting section 11, the first temperature adjustment section 12, and the second temperature adjustment section 13 are held in the second arrangement. When the second period has elapsed in the second arrangement, the main body is rotated again. A nucleic acid amplification reaction is performed by rotation while switching over between the first arrangement and the second arrangement in this manner until completion of a predetermined number of cycles. An operation in which the first arrangement and the second arrangement are switched over once is defined as one cycle.

A period in which the nucleic acid amplification reaction mixture 140 moves from the first region to the second region can be arbitrarily set, but is preferably shorter. For example, the period may be 5 seconds or less, but is more preferably 2 seconds or less, and most preferably 1 second or less.

By performing this thermal cycling treatment using the above-mentioned nucleic acid amplification reaction vessel and elevating type PCR apparatus, the temperature of the reaction mixture in the form of a small liquid droplet can be quickly changed. For example, in shuttle PCR, a period in which the temperature of the reaction mixture is decreased from the high temperature to the set temperature suitable for annealing and elongation or a period in which the temperature of the reaction mixture is increased from the set temperature suitable for annealing and elongation to the high temperature is very short, and therefore, a period in which the temperature of the reaction mixture is outside the temperature range in which annealing and elongation can be performed is also very short.

In the case where RT-PCR is performed, prior to the above-mentioned thermal cycling treatment, a reverse transcription reaction may be performed by heating the second region 112 by the second temperature adjustment section 13 and performing a treatment of the reaction mixture 140 present in the second region 112 at about 42 to 55° C. Specifically, for example, the temperature of the first region 111 of the nucleic acid amplification reaction tube 100 is adjusted to 95° C. by the first temperature adjustment section 12, and the temperature of the second region 112 is adjusted to 42° C. by the second temperature adjustment section 13, and these regions are held in the second arrangement for a predetermined period of time. At this time, the reaction mixture 140 is in the second region 112, and therefore is heated to 42° C. to effect reverse transcription from an RNA to a DNA for a predetermined period of time. Thereafter, the arrangement is switched over to the first arrangement, and thereby the reaction mixture 140 is moved to the first region 111 at 95° C. to inactivate the reverse transcriptase. Then, the above-mentioned thermal cycling treatment may be performed by using the obtained cDNA as a template. The temperature of the reverse transcription reaction and the inactivation temperature of the reverse transcriptase are not particularly limited, however, for example, the temperature of the reverse transcription reaction is generally from about 35 to 65° C., and the inactivation temperature of the reverse transcriptase is generally a temperature at which the DNA polymerase is not inactivated, specifically, it can be arbitrarily selected from the range of about 70 to 105° C.

### (4) Method of Detecting a Plurality of Nucleic Acids in One Nucleic Acid Amplification Reaction Mixture

Hereinafter, a method of detecting a plurality of nucleic acids in one nucleic acid amplification reaction mixture by shuttle PCR using the thermal cycling method according to the invention will be described. Here, a method of detecting first and second nucleic acids in a nucleic acid amplification reaction mixture 140 will be described as an example. As a nucleic acid amplification reaction apparatus, the above-mentioned elevating type PCR apparatus described in (1) and (2) is used, and the thermal cycling treatment is in accordance with the example described in (3). Incidentally, by using this method, three or more nucleic acids can also be independently detected, however, in such a case, the following description explains a method of detecting two nucleic acids among the three or more nucleic acids.

The nucleic acid amplification reaction mixture 140 contains a polymerase, dNTPs, MgCl₂, first and second primer pairs for amplifying first and second nucleic acids, respectively, and a fluorescent label serving as an indicator for amplification of the first and second nucleic acids, and may contain a surfactant and the like as needed. In this method, for the nucleic acid amplification reaction mixture 140, a first thermal cycle is performed at a first temperature and a second temperature, and thereafter, a second thermal cycle is performed at a third temperature and a fourth temperature. The first and third temperatures correspond to the first temperature in (3) suitable for nucleic acid denaturation, and the second and fourth temperatures correspond to the second temperature in (3) suitable for annealing and elongation. The first and second primer pairs are designed so as to satisfy properties as described below.

First, the annealable temperature range of the first primer pair to the first nucleic acid and the annealable temperature range of the second primer pair to the second nucleic acid may overlap each other, but most preferably do not overlap with each other. In the case where the annealable temperature ranges thereof overlap with each other, they overlap by preferably 10° C. or less, more preferably 7° C. or less, further more preferably 5° C. or less, still further more preferably 3° C. or less, and yet still further more preferably 1° C. or less. Here, the annealable temperature range refers to a temperature range in which the amplification of a nucleic acid is observed when the nucleic acid is amplified using each primer pair alone under predetermined conditions.

Further, the optimal annealing temperature of the first primer pair to the first nucleic acid and the optimal annealing temperature of the second primer pair to the second nucleic acid are preferably different from each other, and are preferably different by 1° C. or more, more preferably different by 3° C. or more, and further more preferably different by 5° C. or more. The optimal annealing temperature refers to a temperature at which, when a nucleic acid is amplified by using each primer pair alone under predetermined conditions, the nucleic acid is amplified in the largest amount.

In the case where the annealable temperature range or the optimal annealing temperature is examined, such a temperature can be determined by a thermal cycling treatment using a PCR apparatus, for example, the above-mentioned elevating type PCR apparatus, however, such a temperature is preferably determined by using the same PCR apparatus and under the same conditions as used for amplifying a plurality of nucleic acids in one nucleic acid amplification reaction mixture. The presence or absence of the amplification reaction and the amount of the amplification reaction product can be examined by, for example, measuring the fluorescence brightness of a fluorescent label.

For the nucleic acid amplification reaction mixture 140 containing such primer pairs, first, a first nucleic acid amplification reaction is performed at the first temperature and the second temperature using the thermal cycling method according to the invention. Specifically, the first nucleic acid amplification reaction is performed as follows. First, the temperatures of the first temperature adjustment section and the second temperature adjustment section are set to the first temperature and the second temperature, respectively, and the arrangement of the fitting section 11, the first temperature adjustment section 12, and the second temperature adjustment section 13 is set to the first arrangement. At this time, the nucleic acid amplification reaction mixture 140 is present in the first region 111 and is heated to the first temperature suitable for the nucleic acid denaturation reaction of the first primer pair by the first temperature adjustment section. When the arrangement of the fitting section 11, the first temperature adjustment section 12, and the second temperature adjustment section 13 is switched over to the second arrangement by rotation after an arbitrarily set first period has elapsed, the nucleic acid amplification reaction mixture 140 is present in the second region 112 and is heated to the second temperature suitable for the annealing reaction between the first primer pair and the first nucleic acid by the second temperature adjustment section, and the annealing reaction and the nucleic acid elongation reaction are performed. After an arbitrarily set second period has elapsed, the arrangement is returned to the first arrangement by rotation again. The nucleic acid amplification reaction is performed by rotation while switching over between the first arrangement and the second arrangement at arbitrarily set times. By this operation, the amplification reaction of the first nucleic acid is more efficiently performed than the amplification reaction of the second nucleic acid. In the case where the annealable temperature ranges of the first primer pair and the second primer pair to the nucleic acids do not overlap with each other, substantially only the amplification reaction of the first nucleic acid proceeds.

Subsequently, in order to perform the second nucleic acid amplification reaction at the third temperature and the fourth temperature, the temperatures of the first temperature adjustment section and the second temperature adjustment section are set to the third temperature and the fourth temperature, respectively. In the same manner as the first thermal cycle, first, in the first arrangement, the nucleic acid amplification reaction mixture 140 present in the first region 111 is heated to the third temperature suitable for the nucleic acid denaturation reaction of the second primer pair by the first temperature adjustment section. After an arbitrarily set first period has elapsed, the arrangement of the fitting section 11, the first temperature adjustment section 12, and the second temperature adjustment section 13 is switched over to the second arrangement by rotation to move the nucleic acid amplification reaction mixture 140 to the second region 112, and the temperature of the nucleic acid amplification reaction mixture 140 is decreased to the fourth temperature suitable for the annealing reaction of the second primer pair. After an arbitrarily set second period has elapsed, the arrangement of the fitting section 11, the first temperature adjustment section 12, and the second temperature adjustment section 13 is switched over to the first arrangement by rotation again. The nucleic acid amplification reaction is performed by rotation while switching over between the first arrangement and the second arrangement at arbitrarily set times. By this operation, the amplification reaction of the second nucleic acid is more efficiently performed than the amplification reaction of the first nucleic acid. In the case where the annealable temperature ranges of the first primer pair and the second primer pair to the nucleic acids do not overlap with each other, substantially only the amplification reaction of the second nucleic acid proceeds. In this manner, by the method according to the invention, the first nucleic acid amplification reaction and the second nucleic acid amplification reaction are performed sequentially. Hereinafter, this is referred to as “sequential PCR”.

Here, each of the second temperature and the fourth temperature is set such that a difference from the optimal annealing temperature of the first primer pair or the second primer pair is a predetermined temperature or less. For example, the difference in the temperature may be 5° C. or less, preferably 3° C. or less, and more preferably 1° C. or less, however, it is most preferred that the second temperature and the fourth temperature are set to the optimal annealing temperature of the first primer pair and the optimal annealing temperature of the second primer pair, respectively. It does not matter either the second temperature or the fourth temperature is higher, however, it is preferred that the second temperature is higher than the fourth temperature. That is, it is preferred that among the two primer pairs for amplifying two nucleic acids, a nucleic acid amplification reaction using a primer pair whose optimal annealing temperature is higher is defined as the first nucleic acid amplification reaction and is performed prior to the second nucleic acid amplification reaction.

The first temperature and the third temperature are not particularly limited as long as the first nucleic acid and the first primers, and the second nucleic acid and the second primers can be sufficiently denatured at the temperatures. Further, the first temperature and the third temperature may be the same or different, and for example, may be independently 90° C. or higher, 93° C. or higher, or 95° C. or higher.

When the first nucleic acid amplification reaction is shifted to the second nucleic acid amplification reaction, in the case where the heating temperature by the first temperature adjustment section is changed from the first temperature to the third temperature and the heating temperature by the second temperature adjustment section is changed from the second temperature to the fourth temperature, the temperature changing speeds of the first temperature adjustment section and the second temperature adjustment section at that time can be set arbitrarily, but are preferably faster. For example, the speeds may be 1° C./sec, but is preferably 2° C./sec, more preferably 5° C./sec, and most preferably 10° C./sec.

A period in which the nucleic acid amplification reaction mixture 140 moves from the first region to the second region can be arbitrarily set, but is preferably shorter. For example, the period may be 5 seconds or less, but is preferably 2 seconds or less, and most preferably 1 second or less.

Here, the method according to the invention can also be used in the case of examining whether the first nucleic acid and the second nucleic acid are contained in the nucleic acid amplification reaction mixture 140. That is, for example, when the method according to the invention is used for the nucleic acid amplification reaction mixture 140 in the case where the first nucleic acid is not contained and the second nucleic acid is contained, the amplification of the first nucleic acid does not occur in the first nucleic acid amplification reaction, and the amplification of the second nucleic acid occurs in the second nucleic acid amplification reaction, and therefore, it can be determined at a time with one reaction mixture that the first nucleic acid is not contained and the second nucleic acid is contained.

The number of primer pairs can be arbitrarily determined according to the number of nucleic acids to be detected, and may be 3 or more. When the number of nucleic acids to be detected is represented by n (n is an integer of 1 or more), sequential PCR is performed from the first nucleic acid amplification reaction to the n-th nucleic acid amplification reaction.

Preferably, the fluorescence brightness of the fluorescent label contained in the nucleic acid amplification reaction mixture 140 is monitored during the above-mentioned sequential PCR by the fluorescence detection section.

In the case where the fluorescence brightness to be detected has increased in the first nucleic acid amplification reaction or the second nucleic acid amplification reaction, it can be determined that the amplification of the first nucleic acid or the amplification of the second nucleic acid has occurred. However, it may also be determined that the amplification reaction of the first or second nucleic acid has occurred on the basis that a second derivative obtained from the increased fluorescence brightness has reached the maximum. On the other hand, in the case where the fluorescence brightness has not increased even after performing a predetermined number of cycles, preferably 40 cycles, it is determined that the amplification reaction of a nucleic acid has not occurred.

The time point when the second nucleic acid amplification reaction is started can be arbitrarily set. For example, it has been determined previously that after the first nucleic acid amplification reaction is performed at a predetermined number of cycles, the reaction is shifted to the second nucleic acid amplification reaction. However, the second nucleic acid amplification reaction may be started after it is determined that the amplification reaction of the nucleic acid has occurred in the first nucleic acid amplification reaction, and also the second nucleic acid amplification reaction may be started after it is determined that the amplification reaction of the nucleic acid has not occurred in the first nucleic acid amplification reaction.

By using the method according to the invention described above, the first nucleic acid can be amplified mainly in the first nucleic acid amplification reaction, and the second nucleic acid can be amplified mainly in the second nucleic acid amplification reaction. Therefore, when a test solution for which the presence of the first nucleic acid and the second nucleic acid is tested is used, it can be determined that if the first nucleic acid amplification reaction occurs, the first nucleic acid is present in the test solution, and if the second nucleic acid amplification reaction occurs, the second nucleic acid is present in the test solution.

In this embodiment, as the PCR method, shuttle PCR is used, however, a PCR method (three-stage temperature PCR) in which the temperature is changed in thermal denaturation, annealing, and elongation reactions may be adopted. In this case, in addition to the first and second annealing temperatures, an elongation reaction temperature is set, and after the nucleic acid amplification reaction mixture is maintained at the annealing temperature, it may be maintained at the elongation reaction temperature for a predetermined period of time.

### EXAMPLES

Hereinafter, the invention will be described in more detail by showing Examples, however, the invention is not limited thereto.

### Example 1

An example in which two genes are amplified in one nucleic acid amplification reaction mixture using two types of primer pairs and one type of fluorescent dye with the above-mentioned elevating type PCR apparatus will be described. As the PCR method, shuttle PCR in which annealing and elongation reactions are performed at the same temperature was selected. Hereinafter, the annealing temperature means a temperature at which the annealing and elongation reactions are performed.

### (1) Determination of Annealable Temperature Range and Annealing Temperature

As a PCR apparatus of Comparative Example, StepOnePlus (Applied Biosystems) was used, and in the method according to the invention, the above-mentioned elevating type PCR apparatus was used. Unlike the elevating type PCR apparatus, StepOnePlus is a related art apparatus configured such that a tube in which a nucleic acid amplification reaction mixture is placed is fitted in a thermal block, and the temperature of the nucleic acid amplification reaction mixture is increased and decreased by repeatedly heating and cooling the thermal block.

The reaction mixture contained two genes and a reagent for nucleic acid amplification, and one type of fluorescent dye was used.

As the genes to be amplified, β-actin and G1-b were used.

First, in order to determine the annealable temperature range and the annealing temperature for a nucleic acid amplification reaction using StepOnePlus, under the following conditions, the amplification reactions of the two genes were performed separately. The amount of the reaction mixture was set to 10 μL, and 10⁴ copies of each gene were amplified.

TABLE 1

| | Temperature | Period | Cycle number |
|---|---|---|---|
| HS | 95° C. | 1 min | |
| Thermal | 95° C. | 1 sec | 50 cycles |
| denaturation | | | |
| Annealing and | 40 to 70° C. | 10 sec | |
| elongation | | | |

The sequences of the primer pairs and probes used are as follows.

(1) Primer Pair and Probe used for β-actin
β-actin RNA2 Forward primer:
(SEQ ID NO: 1)
5-AGCCTCGCCTTTGCCGA-3
β-actin RNA2 Reverse primer:
(SEQ ID NO: 2)
5-CTGGTGCCTGGGGCG-3
β-actin RNA2 probe:
(SEQ ID NO: 3)
FAM-CCGCCGCCCGTCCACACCCGCC-TAMRA
(2) Primer Pair and Probe used for G1-b
COG1 Forward primer:
(SEQ ID NO: 4)
5-CGYTGGATGCGNTTYCATGA-3
COG1 Reverse primer:
(SEQ ID NO: 5)
5-CTTAGACGCCATCATCATTYAC-3
RING1-TP(b) probe:
(SEQ ID NO: 6)
FAM-AGATCGCGGTCTCCTGTCCA-MGB

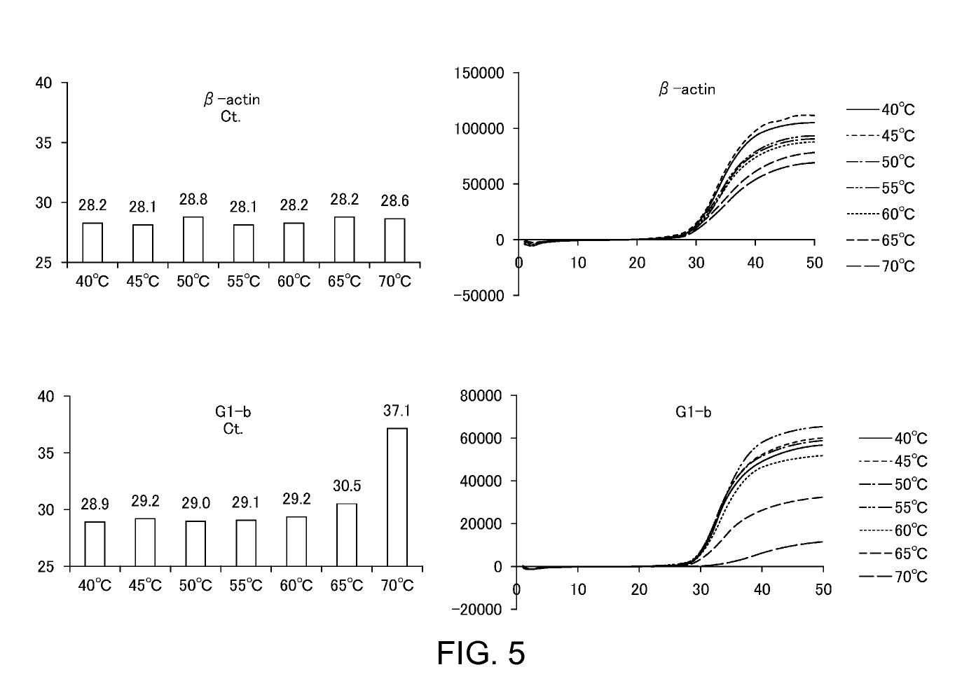

A relationship between the amplification of each of the two genes and the temperature is shown in FIG. 5. In each bar graph, the vertical axis represents the cycle number at which amplification starts to be detected (Ct value) and the horizontal axis represents the annealing temperature. In the line graph, the vertical axis represents the fluorescence brightness and the horizontal axis represents the cycle number. The numerical value represents an average of two tests. In StepOnePlus (related art), amplification was observed at 40 to 70° C. for both genes. That is, in StepOnePlus, the annealable temperature ranges of the respective primer pairs were both from 40 to 70° C.

Subsequently, in order to determine the annealable temperature range and the annealing temperature for a nucleic acid amplification reaction using the elevating type PCR apparatus, under the following conditions, the amplification reactions of the two genes were performed separately. The amount of the reaction mixture was set to 1.6 μL, and 10⁴ copies of each gene were amplified.

TABLE 2

| | Temperature | Period | Cycle number |
|---|---|---|---|
| HS | 98° C. | 1 min | |
| Thermal | 98° C. | 5 sec | 50 cycles |
| denaturation | | | |
| Annealing and | 40 to 70° C. | 10 sec | |
| elongation | | | |

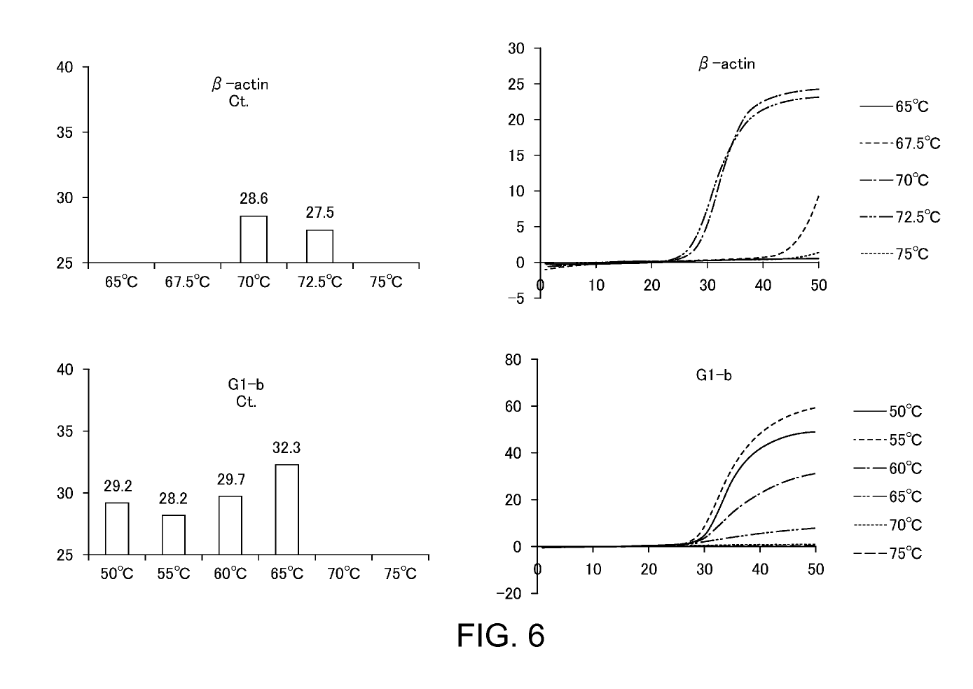

A relationship between the amplification of each of the two genes and the temperature is shown in FIG. 6. In each bar graph, the vertical axis represents the cycle number at which amplification starts to be detected (Ct value) and the horizontal axis represents the annealing temperature. In each line graph, the vertical axis represents the fluorescence brightness and the horizontal axis represents the cycle number. The numerical value represents an average of two tests. In the elevating type PCR apparatus used in the method according to the invention, the temperature range in which annealing and elongation can be achieved was narrower than in the related art method, and the annealable temperature ranges did not overlap with each other. Further, the most efficient annealing and elongation temperatures (optimal annealing temperatures) for the two genes were 72.5° C. and 55° C., respectively.

### (2) Comparison Between Example According to Invention and Comparative Example

By using the elevating type PCR apparatus (the invention) and StepOnePlus (related art), the amplification of the above-mentioned two genes was sequentially performed in one reaction mixture (sequential PCR). The amount of the reaction mixture was set to 1.6 μL for the elevating type PCR apparatus and 10 μL for StepOnePlus in the same manner as in (1), and 10⁴ copies of each gene were amplified. The conditions for sequential PCR are as follows.

TABLE 3
Conditions for Sequential PCR in StepOnePlus

| | | Temperature | Period | Cycle number |
|---|---|---|---|---|
| | HS | 95° C. | 1 min | |
| First nucleic | Thermal | 95° C. | 1 sec | 40 |
| acid | denaturation | | | cycles |
| amplification | Annealing and | 72.5° C. | 10 sec | |
| reaction | elongation | | | |
| Second | Thermal | 95° C. | 1 sec | 40 |
| nucleic acid | denaturation | | | cycles |
| amplification | Annealing and | 55° C. | 10 sec | |
| reaction | elongation | | | |

TABLE 4
Conditions for Sequential PCR in Elevating type PCR Apparatus

| | | Temperature | Period | Cycle number |
|---|---|---|---|---|
| | HS | 98° C. | 1 min | |
| First nucleic | Thermal | 98° C. | 5 sec | 40 |
| acid | denaturation | | | cycles |
| amplification | Annealing and | 72.5° C. | 10 sec | |
| reaction | elongation | | | |
| Second | Thermal | 98° C. | 5 sec | 40 |
| nucleic acid | denaturation | | | cycls |
| amplification | Annealing and | 55° C. | 10 sec | |
| reaction | elongation | | | |

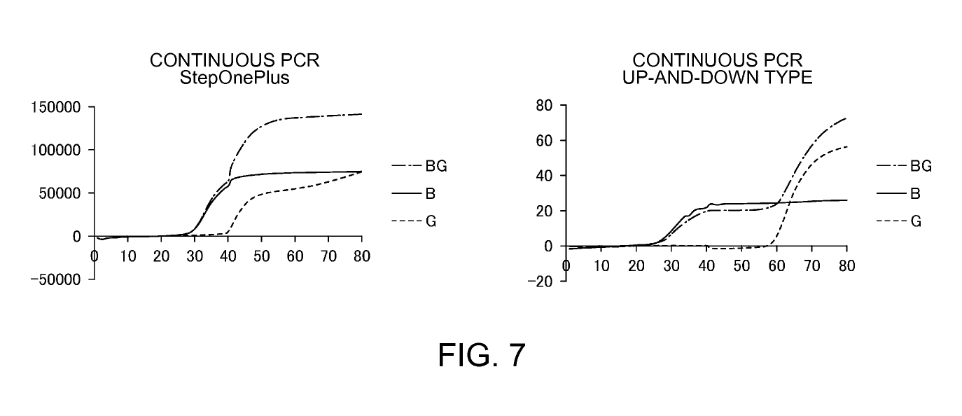

For both of the StepOnePlus (related art) and the elevating type PCR apparatus (the invention), the amplification of the above-mentioned two genes is shown in FIG. 7. In each line graph, the vertical axis represents the fluorescence brightness and the horizontal axis represents the cycle number. The numerical value represents an average of two tests. B indicates the fluorescence brightness derived from the amplification of β-actin, G indicates the fluorescence brightness derived from the amplification of G1-b, and BG indicates the total fluorescence brightness derived from the amplification of β-actin and G1-b.

As shown in FIG. 7, in the elevating type PCR apparatus, the fluorescence brightness stopped increasing at the time point when the first nucleic acid amplification reaction was shifted to the second nucleic acid amplification reaction, and thereafter, the fluorescence brightness started increasing again. This indicates that by changing the annealing temperature from the first annealing temperature to the second annealing temperature when the first nucleic acid amplification reaction was shifted to the second nucleic acid amplification reaction, the amplification of β-actin stopped and the amplification of G1-b started. On the other hand, in StepOnePlus of the related art, the fluorescence brightness did not stop increasing at the time point when the first nucleic acid amplification reaction was shifted to the second nucleic acid amplification reaction. This indicates that because the temperature at which the amplification of β-actin occurs and the temperature at which the amplification of G1-b occurs overlapped with each other, a plurality of genes could not be amplified independently.

In this manner, with the use of the method according to the invention, by changing the annealing temperature (or annealing and elongation temperature) according to the primer pair, a plurality of nucleic acids could be independently amplified and detected in one reaction mixture. On the other hand, in StepOnePlus (related art), different genes could not be independently amplified or detected.

As described above, in the method according to the invention, it is only necessary to prepare one fluorescence detection system and one reaction mixture, and therefore, the operation is simple and the cost can be reduced. Further, a dispensing operation is not needed, and therefore, an error due to the dispensing operation does not occur. Further, the inhibition of amplification reactions due to interference among respective primers caused by simultaneous amplification of a plurality of nucleic acids, which was a problem when a plurality of nucleic acids are amplified in one reaction mixture, does not occur. In addition, in the case of monitoring fluorescence brightness, the presence or absence of the amplification of a nucleic acid can be determined by a change in the fluorescence brightness, and therefore, an additional melting curve analysis or an electrophoresis operation is not needed, and thus, the determination can be achieved in a short time.

The entire disclosure of Japanese Patent Application No. 2014-222570, filed Oct. 31, 2014 is expressly incorporated by reference herein.

What is claimed is:

1. A nucleic acid amplification reaction apparatus, comprising:
a fitting section capable of fitting a nucleic acid amplification reaction vessel filled with a nucleic acid amplification reaction mixture and a liquid which has a specific gravity smaller than that of the nucleic acid amplification reaction mixture and is immiscible with the nucleic acid amplification reaction mixture;
a first temperature adjustment section which adjusts the temperature of a first region of the nucleic acid amplification reaction vessel;
a second temperature adjustment section which adjusts the temperature of a second region of the nucleic acid amplification reaction vessel; and
a driving mechanism which switches over between a first arrangement in which the first region is located lower than the second region in the direction of the gravitational force and a second arrangement in which the second region is located lower than the first region in the direction of the gravitational force, wherein
for the nucleic acid amplification reaction mixture, which contains a first primer pair for amplifying a first nucleic acid and a second primer pair for amplifying a second nucleic acid, and in which the annealable temperature range of the first primer pair to the first nucleic acid and the annealable temperature range of the second primer pair to the second nucleic acid overlap by 10° C. or less,
a first thermal cycle is performed by adjusting the temperature of the first region to a first temperature and adjusting the temperature of the second region to a second temperature which is lower than the first temperature, and thereafter,
a second thermal cycle is performed by adjusting the temperature of the first region to a third temperature and adjusting the temperature of the second region to a fourth temperature which is lower than the third temperature and different from the second temperature.

2. The nucleic acid amplification reaction apparatus according to claim 1, wherein the second temperature is higher than the fourth temperature.

3. The nucleic acid amplification reaction apparatus according to claim 1, wherein a temperature difference between the second temperature and the optimal annealing temperature of the first primer pair to the first nucleic acid is 5° C. or less.

4. The nucleic acid amplification reaction apparatus according to claim 1, wherein a temperature difference between the second temperature and the optimal annealing temperature of the first primer pair to the first nucleic acid is 1° C. or less.

5. The nucleic acid amplification reaction apparatus according to claim 1, wherein a temperature difference between the fourth temperature and the optimal annealing temperature of the second primer pair to the second nucleic acid is 5° C. or less.

6. The nucleic acid amplification reaction apparatus according to claim 1, wherein a temperature difference between the fourth temperature and the optimal annealing temperature of the second primer pair to the second nucleic acid is 1° C. or less.

7. The nucleic acid amplification reaction apparatus according to claim 1, wherein
the first thermal cycle is a thermal cycle in which a two-stage temperature change between the first temperature and the second temperature is repeated, and
at the second temperature, the first nucleic acid is amplified and the second nucleic acid is not amplified.

8. The nucleic acid amplification reaction apparatus according to claim 1, wherein
the second thermal cycle is a thermal cycle in which a two-stage temperature change between the third temperature and the fourth temperature is repeated, and
at the fourth temperature, the second nucleic acid is amplified and the first nucleic acid is not amplified.

9. A nucleic acid detection method, which uses a nucleic acid amplification reaction apparatus including:
a fitting section capable of fitting a nucleic acid amplification reaction vessel filled with a nucleic acid amplification reaction mixture and a liquid which has a specific gravity smaller than that of the nucleic acid amplification reaction mixture and is immiscible with the nucleic acid amplification reaction mixture;
a first temperature adjustment section which adjusts the temperature of a first region of the nucleic acid amplification reaction vessel;
a second temperature adjustment section which adjusts the temperature of a second region of the nucleic acid amplification reaction vessel; and
a driving mechanism which switches over between a first arrangement in which the first region is located lower than the second region in the direction of the gravitational force and a second arrangement in which the second region is located lower than the first region in the direction of the gravitational force, wherein
for the nucleic acid amplification reaction mixture, which contains a first primer pair for amplifying a first nucleic acid and a second primer pair for amplifying a second nucleic acid, and in which the annealable temperature range of the first primer pair to the first nucleic acid and the annealable temperature range of the second primer pair to the second nucleic acid overlap by a predetermined temperature or less,
the method comprises:
performing a first thermal cycle by setting the temperature of the first region to a first temperature and setting the temperature of the second region to a second temperature which is lower than the first temperature; and
performing, after the first thermal cycle, a second thermal cycle by setting the temperature of the first region to a third temperature and setting the temperature of the second region to a fourth temperature which is lower than the third temperature and different from the second temperature.

10. The nucleic acid detection method according to claim 9, wherein the annealable temperature ranges overlap by 5° C. or less.

11. The nucleic acid detection method according to claim 9, wherein the annealable temperature ranges do not overlap with each other.

12. The nucleic acid detection method according to claim 9, wherein the optimal annealing temperature of the first primer pair to the first nucleic acid and the optimal annealing temperature of the second primer pair to the second nucleic acid are different by 3° C. or more.

13. The nucleic acid detection method according to claim 9, wherein the nucleic acid amplification reaction apparatus further includes a fluorescence detection section, and the fluorescence detection section monitors fluorescence emitted from the nucleic acid amplification reaction mixture while the nucleic acid amplification reaction apparatus is performing the first thermal cycle and the second thermal cycle.
